(19) United States
(12) Patent Application Publication
(10) Pub. No.: US 2006/0020314 A1
(43) Pub. Date: Jan. 26, 2006

(54) Systems and methods for characterizing leads

(75) Inventor: Jeffrey P. Bodner, Roseville, MN (US)

Correspondence Address: SCHWEGMAN, LUNDBERG, WOESSNER & KLUTH, 1600 TCF TOWER, 121 SOUTH EIGHT STREET, MINNEAPOLIS, MN, 55402, US

(73) Assignee: Cardiac Pacemakers, Inc.

(21) Appl. No.: 10/898,455

(22) Filed: Jul. 23, 2004

(51) Int. Cl. A61N 1/05

(52) U.S. Cl. 607/116

(57) ABSTRACT

Indicia represent characteristics of an implantable lead. The characteristics include at least one of stiffness, bending stiffness, torsional stiffness, weight, mass per unit length, electrical properties, lubricity, mechanical resistance, and pushability. The indicia include a plurality of alpha-numeric characters and/or graphics, which represent a corresponding plurality of lead characteristics.

### TECHNICAL FIELD

Systems and methods for characterizing implantable leads for conducting electrical signals, and, more particularly, systems and methods for characterizing leads that conduct electrical signals to and from the heart.

### TECHNICAL BACKGROUND

Pacemaker leads represent the electrical link between the pulse generator and the heart tissue, which is to be excited and/or sensed. These pacemaker leads include single or multi-conductors that are connected to an electrode in an electrode assembly at an intermediate portion or distal end of a pacing lead. A connector is included at the proximal end to form the electrical connection with the pulse generator, such as a pacemaker. The lead has particular electrical characteristics that are adapted for use with a particular pulse generator and the type of therapy provided by the pulse generator to the heart tissue. Leads are discussed in U.S. Pat. Nos. 6,701,191; 6,672,895; 6,671,560; 6,606,522; 6,584,363; 6,584,362; 6,577,904; 6,574,514; 6,574,512; 6,564,107; and 6,212,434, which are hereby incorporated by reference for any purpose.

To implant the lead within the patient, the lead is fed intravenously toward the heart. The lead may be implanted within or travel through complex or tortuous vasculature. The lead may also need to travel through vasculature having increasingly smaller diameters. Accordingly, the lead has particular mechanical characteristics that relate to the lead's size and flexibility.

There is a need for a system to efficiently identify the characteristics of a lead within and between the lead development community, the medical practitioners, sales departments, and the patient community. There exists difficulty in communicating lead characteristics between people involved with leads. There is also a need to standardize the system used to identify leads while minimizing engineering terminology to make the system more acceptable outside the engineering community.

### SUMMARY

Systems and methods for characterizing leads include indicia that represent characteristics of a lead. In an option, the lead is an implantable lead. The indicia are associated with a lead to provide a means of identifying the characteristics of the lead. Characteristics of a lead include operational characteristics of the lead. In an option, the operational characteristics include performance features related to sensing of patient health statistics, delivery of therapeutic signals and/or insertion of the lead into a patient's body.

The lead includes a body and electrical conductor within the body. In an option, the indicia are connected to the body. In an option, the indicia are on the body. In an option, the indicia are covered by a coating. The coating includes therapeutic agent in an option.

Several options for the indicia are as follows. The indicia represent at least one mechanical property of the lead. The mechanical property includes at least one of bending stiffness, torsional stiffness, weight, and mass per unit length. The indicia can also represent an electrical property. The indicia include a plurality of alpha-numeric characters, which represent a corresponding plurality of lead characteristics. The indicia include a graphic representation of the characteristics of the lead. The graphic representation includes a grid. The grid is a three-dimensional grid having a plurality of locations each corresponding to a specific characteristic of the lead. The graphic representation includes a bar graph. The bar graph includes a bar representing a lead characteristic variable, and the bar has a height representing a value of the lead characteristic variable. The bar includes shading or coloring representing the lead characteristic.

An apparatus of an embodiment includes a display and indicia associated with the display. The indicia represent characteristics of an implantable lead. In an option, the display includes an electronic device. In an option the electronic device includes a computer network. In an option, the computer network includes a unit for entering a request for the indicia, and a server operably connected to the unit and adapted to provide the indicia to the unit. The server is adapted to provide additional data associated with the indicia. In an option, the electronic device includes a computer readable media storing the indicia and data related to the characteristics denoted by the indicia.

A method assigns indicia to an implantable lead. The method includes providing characteristics of a lead type, assigning indicia representing the characteristics of the lead type, associating the assigned indicia to leads of the lead type. Several options for the indicia are as follows. Associating indicia includes imprinting the indicia on the lead. Associating indicia includes attaching the indicia to the lead. Assigning indicia includes assigning an alpha-numeric code for the indicia. Associating indicia includes storing the indicia in a computer readable media. Associating indicia includes providing access to the computer readable media. Providing access to the media includes providing access to the indicia through a computer network. Associating indicia includes storing additional data relating to the indicia and the lead type in the computer readable media. Providing access to the media includes requesting the user to select the indicia before providing access to the additional information.

The indicia system and method provides a user, such as a physician, with an readily understood indicator of how a lead will react during insertion into a patient. In an option, the indicia system will provide the user with an understanding of the physical characteristics of the lead. This information allows a user to determine if the correct lead is being used so that the patient receives the proper lead and the physician is implanting a lead that has the favorable characteristics for the patient physiology and physician preferences.

This Summary is an overview of some of the teachings of the present application and is not intended to be an exclusive or exhaustive treatment of the present subject matter. Further details about the present subject matter are found in the detailed description and appended claims. Other aspects will be apparent to persons skilled in the art upon reading and understanding the following detailed description and viewing the drawings that form a part thereof, each of which are not to be taken in a limiting sense. The scope of the present invention is defined by the appended claims and their equivalents.

### BRIEF DESCRIPTION OF THE DRAWINGS

FIG. 1 is a diagram illustrating a lead system constructed according to one embodiment.

FIG. 2 is an enlarged, partial view of a lead according to one embodiment.

FIG. 3 is an enlarged partial view of a lead according to one embodiment.

FIG. 4 is an enlarged partial view of a lead according to one embodiment.

FIG. 5 is an enlarged partial view of a lead according to one embodiment.

FIG. 6 is an enlarged partial view of a lead according to one embodiment.

FIG. 7 is an enlarged partial view of a lead according to one embodiment.

FIG. 8 is an enlarged partial view of a lead according to one embodiment.

FIG. 9 is a schematic view of a system according to one embodiment.

### DESCRIPTION OF THE EMBODIMENTS

The following detailed description of the present invention refers to subject matter in the accompanying drawings which show, by way of illustration, specific aspects and embodiments in which the present subject matter may be practiced. These embodiments are described in sufficient detail to enable those skilled in the art to practice the present subject matter. Other embodiments may be utilized and structural, logical, and electrical changes may be made without departing from the scope of the present subject matter. References to “an”, “one”, or “various” embodiments in this disclosure are not necessarily to the same embodiment, and such references contemplate more than one embodiment. The following detailed description is, therefore, not to be taken in a limiting sense, and the scope is defined only by the appended claims, along with the full scope of legal equivalents to which such claims are entitled.

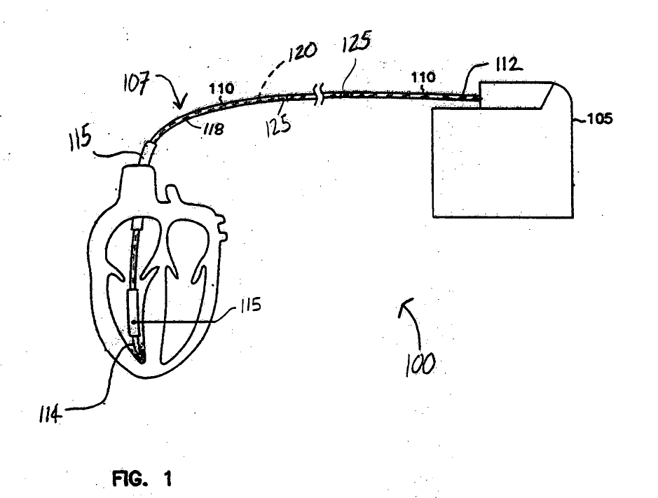

An implantable system 100 including an electrical stimulator 105 is illustrated in FIG. 1. It should be noted that the system 100 is suitable for use with implantable electrical stimulators, such as, but not limited to, pulse generators, neuro-stimulators, skeletal stimulators, central nervous system stimulators, or stimulators for the treatment of pain. The implantable device 100 includes an implantable lead 107 that includes body 110 and at least one elongate conductor 120 extending within the lead body 110. The lead body 110 typically surrounds the conductor 120 to insulate the conductor from the surrounding environment, such as tissue, and to target any electrical signal to or from a specific location of a person's body. The lead 107 extends from a proximal end 112 to a distal end 114. The proximal end 112 of the lead is electrically coupled with the electrical stimulator 105, for example, with a connector. Lead 107 is adapted to carry various signals associated with sensing and therapy for cardiac pacing and defibrillation, for example, bradycardia, tachycardia, tachyarrhythmia, and left ventricular pacing.

In an embodiment, the electrical stimulator 105 is adapted to be implanted with a person's body and thus the entire device 100 is adapted to be implanted in a person's body. In one option, the electrical stimulator 105 is a pulse sensor and generator that contains electronics to sense various electrical signals of the heart and also produce current pulses for delivery to the heart. The pulse sensor and generator also contains electronics and software necessary to detect certain types of health problems, such as arrhythmias, and to provide therapies for them.

The implantable device 100 further includes, in one option, one or more electrodes 115. The one or more electrodes 115 are each electrically coupled with the conductor 120, which in turn electrically couples the electrode 115 to a signal generation circuit or a detection circuit in implant 105. The electrode 115 allows for electrical signals to be delivered to the tissue from the electrical stimulator 105. The implantable device 100 further includes, in one option, features to allow the lead body to be fixated within a patient. For example, in one option, the lead body includes passive fixation features, such as one or more tines. In another option, the lead body includes an active fixation assembly, such as a fixation helix.

Indicia 125 are associated with the lead 107. The indicia 125 provide an indicator of the characteristics and/or properties of the lead 107. Examples of lead characteristics include, but are not limited to, stiffness, weight, length, diameter, electrical capacity, lubricity, or coating type. Other lead characteristics are fixation features. Stiffness includes, but is not limited to, torsional stiffness and bending stiffness. Electrical capacity includes, but is not limited to, impedance. Coating type includes, but is not limited to, medications or biomedical coatings that provide further therapy to the person receiving the implant. Mass per unit length is a further lead characteristic. Lubricity is a further lead characteristic. Pushability of over-the-wire leads is a further lead characteristic. In an option, the indicia 125 are positioned on an outer surface of lead body 110. Lead 107 may include a covering, which is inert with respect to the environment in which it is implanted, over the indicia 125. The covering does not interfere with the indicia's ability to convey information regarding the characteristics of the lead. In an option, the covering is transparent over the indicia 125. In an option, indicia 125 are removably attached to the lead 107. In an option, indicia 125 are displayed on materials describing or promoting lead 107.

In the embodiment shown in FIG. 1, there is a plurality of indicia 125. Each indicia 125 is the same when the indicia represent characteristics of the whole lead 107. That is the indicia 125 denotes a composite performance grade indicating the operational characteristics of the entire lead. In another option, indicia 125 represent characteristics of portions of lead 107. That is, the left indicia as shown in FIG. 1 represents characteristics of the left portion of lead 107, e.g., to the left of the break line remote from the signal generator/signal analysis device 105. The right indicia as shown in FIG. 1 represents characteristics of the right portion of lead 107, e.g., to the right of the break line adjacent the signal generator/signal analysis device 105.

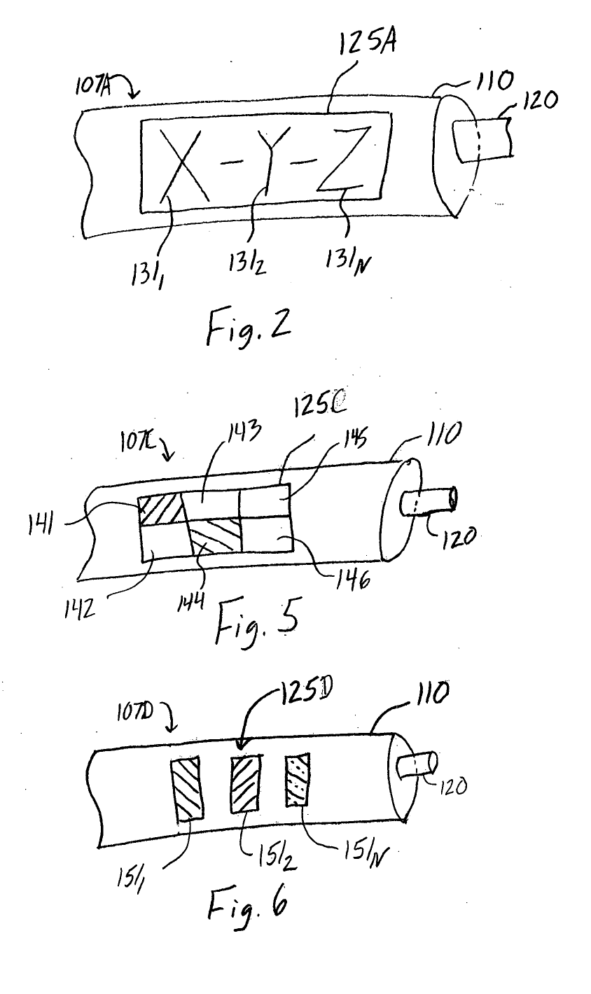

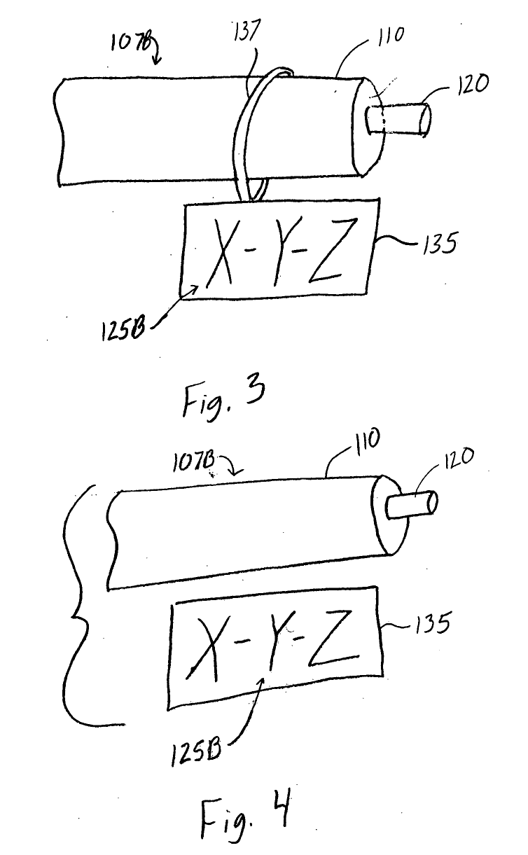

FIG. 2 shows an enlarged, partial length of lead 107A including lead body 110 and conductor 120. Indicia 125A are associated with lead 107A. The same elements throughout the figures of the present application are designated by the same reference number. In some embodiments, alphabetic characters are added as suffixes to the reference numbers thereby delineating between the figures. Indicia 125A include a plurality of alpha-numeric characters 131₁, 131₂, . . . 131_{N}. Each character 131₁, 131₂, . . . 131_{N} represents one characteristic of the lead 107A. At least one characteristic is a variable. The value of the characters 131₁, 131₂, . . . 131_{N} are selected to represent the specific ones of the variable characteristics. In an embodiment, there are three characteristics that are being indicated by indicia 125A. The first character 131₁ represents bending stiffness of the lead 107A. In this example, bending stiffness is a lead variable that has two potential values, low and high. First character 131₁ accordingly has two different potential values representing low and high respectively. First character 131₁ is a one for low or a two for high in an embodiment. First character 131₁ is the alphabetic character “L” for low or the alphabetic character “H” for high in an embodiment. Other representative characters may be selected to represent these characteristics and are within the scope of embodiments of the present invention. The second character 131₂ represents the weight of the lead 107A. In this example, weight is a lead variable that has five potential values over a range. Second character 131₂ accordingly has five different potential values representing a value in the weight range. Second character 131₂ is selected from a group that represents the range of five values. In an embodiment, the group consists of the numeric characters “1”, “2”, “3”, “4”, and “5”. The third character 131₃ represents the torsional stiffness of the lead 107A. In this example, torsional stiffness is a lead variable that has three potential values over a range. Third character 131₃ is selected from a group that represents the range of three values. In an embodiment, the third character group consists of the numeric characters “1”, “2”, or “3”. In an option, the indicia 125A would be displayed as H-5-1. Such indicia represent high bending stiffness, the heaviest lead, and the lowest torsional stiffness. While these examples describe bending stiffness, weight, and torsional stiffness, it will be recognized that The indicia described herein are adaptable to other unique physical characteristics of a lead.

FIGS. 3 and 4 show an enlarged, partial length of lead 107B including lead body 110 and conductor 120. Indicia 125B are associated with lead 107B. In an option, indicia 125B include alpha-numeric characters representing characteristics of lead 107B as described herein. The indicia 125B are displayed on a base 135. In an option, base 135 is printed material on which indicia 125B are shown and visible to a user. An attachment device 137 secures base 135 to the lead body 110 as shown in FIG. 3. Attachment 137 is a plastic strip or fiber device that ties to lead body 110. Attachment 137 is removed before implant of the lead 107B into a patient. FIG. 4 shows the base 135 associated with the lead 107B without an attachment. The base 135 provides a user with the indicia 125B, which convey the characteristics of the lead 107B to the user, e.g., the medical personnel.

FIG. 5 shows an enlarged, partial length of lead 107C including lead body 110, conductor 120 and indicia 125C. Indicia 125C include a grid that has, in an option, horizontal and vertical lines to define areas 141-146 in the grid. The areas 141-146 within the grid are selectively darkened or colored to indicate the properties of the lead. The grid is read vertically in an embodiment to define three features of the lead 107C, i.e., vertically aligned area pairs 141/142, 143/144, and 145/146 indicate the value of one feature, respectively. In an option the areas 141-146 each have two possible values. Accordingly, each area pair has four possible values with the areas 141-146 either being undarkened, i.e., a zero value, or darkened, i.e., a one value, in a binary system. In the illustrated example of FIG. 5, the first feature of lead 107C is indicated with pair 141/142 having area 141 darkened and area 142 undarkened, i.e., a 01 value. The second feature of lead 107C is indicated with pair 143/144 having area 143 undarkened and area 144 darkened, i.e., a 01 value. The third feature of lead 107C is indicated with pair 145/146 having areas 145 and 146 undarkened, i.e., a 00 value. In an option, the areas 141-146 are cross-hatched to indicate a value of a lead characteristic. In an option, the areas 141-146 are colored to indicate a value of a lead characteristic. In an option, each of areas 141-146 represents a binary value for six different lead characteristics.

FIG. 6 shows an enlarged, partial length of lead 107D including lead body 110, conductor 120 and indicia 125D. Indicia 125D include a graph that has a plurality of vertical segments 151₁, 151₂, . . . 151_{N}. Each of the segments 151₁, 151₂, and 151_{N} are selectively darkened or colored to indicate a characteristic of the lead 107D. The characteristics of lead 107D are denoted by the combination of vertical segments 151₁, 151₂, and 151_{N}.

FIG. 7 shows an enlarged, partial length of lead 107E including lead body 110, conductor 120 and indicia 125E. Indicia 125E include a graph that has, in an option, a plurality of vertical segments 161₁, 161₂, and 161_{N}. Each of the segments 161₁, 161₂, and 161_{N} are respectively set at a height to indicate a characteristic of the lead 107E. In an option, the height of at least one of segments 161₁, 161₂, and 161_{N} indicates a particular value of a characteristic of lead 107E. For example, indicia 125E indicates the following characteristics by segment: the first segment 161₁ indicates the type of connector on lead 107E; the second segment 161₂ indicates the weight of lead 107E; and the third segment 161_{N} indicates the stiffness of lead 107E. Other characteristics of a lead are illustratable using similar and/or additional segments.

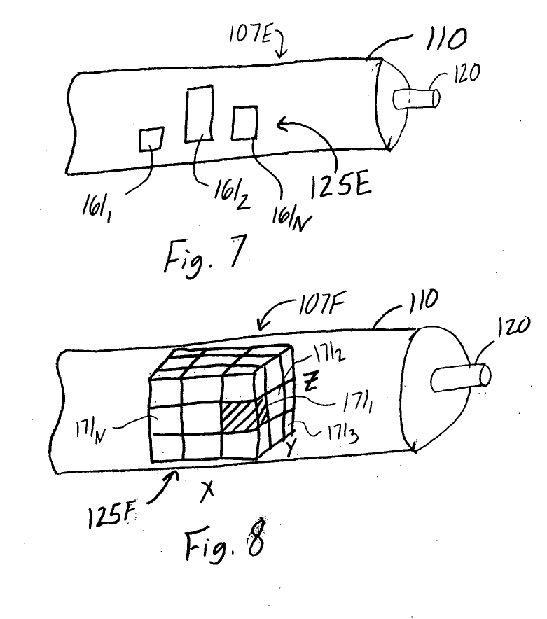

FIG. 8 shows an enlarged, partial length of lead 107F including lead body 110, conductor 120 and indicia 125F. Indicia 125F includes a three dimensional graphic extending in X, Y, and Z directions and has three visible surfaces. Each of the three visible surfaces is divided into distinct parts or surface portions 171₁, 171₂, 171₃, . . . 171_{N}. Each surface portion 171₁, 171₂, 171₃, . . . 171_{N} is adapted to indicate characteristics of lead 107F. In the illustrated embodiment, a single surface portion 171₁ indicates the characteristics of lead 107F. In an option, a plurality of surface portions is shaded to designate lead characteristics. For example, each side of the indicia 125F is dedicated to one characteristic and at least one of the plurality of surface portions on a specific side indicate a specific value of the lead characteristic associated with that specific side.

The above description provides exemplary embodiments for conveying information regarding characteristics of an implantable lead for medical applications. There remains a need for an effective system to disseminate the definitions of the indicia. That is, some of the indicia described may not be readily understood by users of this information conveying system until indicia are widely accepted into standard practice.

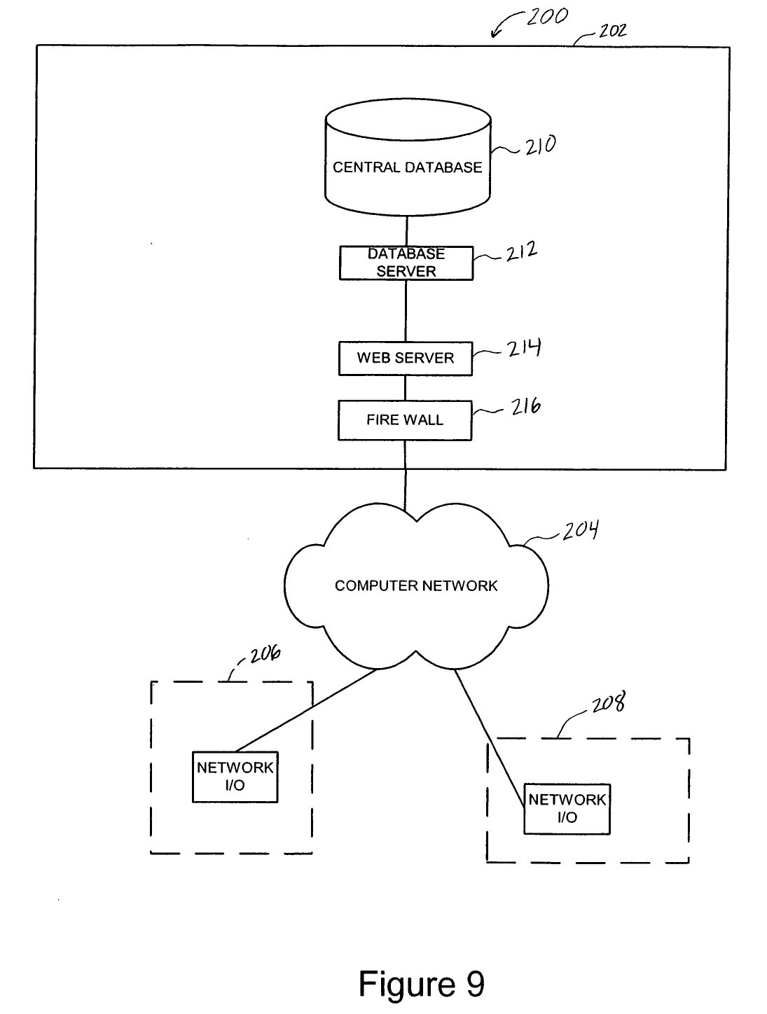

FIG. 9 shows an embodiment of system 200 for conveying information to familiarize a user with regard to the meaning of the indicia. System 200 includes a central information system 202 connected over a network 204, such as an electronic or computer network, to a first interface device 206 and a second interface device 208. The computer network 204 is a global computer network, such as the internet, in an embodiment. The computer network 204 is a wide area network (WAN), local area network (LAN) or other similar network in an embodiment. Computer network 204 includes a wireless network in an embodiment. Information system 202 is adapted to communicate with the interface devices 206 and 208 through the network 204. Such communication requires a communications interface between the components of system 200. Examples of a communication interface include protocols such as File Transfer Protocol (FTP) and other network communication standards such as html, xml, or other codes compatible with network interfaces such a web browsers (Internet Explorer, Netscape, etc.). The communications over the computer network can be made using known secure means such as using the Secure Sockets Layer (SSL) Protocol or using other forms of encryption. Interface devices 206 and 208 each include a network input/output unit to allow the interface device to communicate with the network 204. The network input/output units may not be the same in each interface device. Accordingly, the first interface device 206 may be a personal computer connected to the internet through a modem. The second interface 208 device may be a portable electronic device such as a mobile telephone of personal data assistant. Interface devices 206 and 208 further include user input/output devices such as keyboards, number pads, view screens, and other typical devices.

System 202 includes a central database 210 that stores data related to the indicia for lead characteristics. The data includes the code and/image of the indicia and specific data related to the lead as indicated by the indicia. In an option, the specific data includes a detailed specification list of the lead. In an option, the specific data includes manufacturing data. In an example, the indicia 125A are associated with a lead to denote its characteristics. The central database 210 stores the indicia value and the lead characteristics represented by each specific indicia. In a further example, one of the graphic indicia 125C-125F is used to denote lead characteristics. Central database 210 stores each graphic indicia and lead characteristics denoted by indicia. System 202 further includes a database server 212 to provide access to the central database 210. The database server 212 in an embodiment use Microsoft (Trade Mark) SQL Server 2000. Database server 212 communicates through a web server 214 and firewall 216 with the computer network 204. The web server 214 is an Apache web server running Perl scripts and JavaScript and use stored static HTML templates and dynamic page generation code. The Apache servers also use Mason (Trade Mark) to allow mix of Perl and HTML to be used.

In use, system 200 allows a user through interface devices 206 and 208 to access data that explains the indicia 125A-125F. A user accesses the central information system 202 for example by visiting a web page or logging into a private computer network. The user may enter the indicia into an input of the central information system 202, which will in turn display or download data related to the lead identified by the indicia. If the indicia are a graphic, then the central information system 202 may provide a menu, icons, or thumbnail of all indicia for the users to choose from. The user will select at least one of the indicia for to access additional data. Selection is made by clicking on the menu item, icon, or thumbnail. Selection can be further automated such that data is displayed as the user passes a cursor over one of the menu items, icons, or thumbnails. The central information system further includes a contact interface that allows the user to contact support or sales personnel through voice or email.

Central information system 202 is stored on a computer readable medium in an option. The computer readable medium includes read only memory, random access memory, static memory, EEPROM, optically read memory, compact disks, DVD formatted disks, magnetic media, and other computer readable medium known in the computer readable media art. When the central information system 202 is stored on portable computer readable media, then the data associated with the indicia 125A-125F can be taken to and left with users so that a user need not access central information system 202 through computer network 204.

Advantageously, the system described herein allows a user, such as a physician, research and developer, marketer, etc., to effectively communicate regarding lead characteristics. The characteristics relate to the lead, per se. Such characteristics relate to implantation of the lead in a patients body and include diameter, length, stiffness, lubricity, and pushability, which are directly related to inserting the lead into a patient's body. Other characteristics related to the lead include electrical characteristics. Once the system described herein users of leads will be able to accurately communicate their needs and preferences with regard to leads so that patients receive improved products and care.

It is to be understood that the above description is intended to be illustrative, and not restrictive. It will be understood that the examples and illustrations described herein are adaptable to any characteristic of a lead in need of description to facilitate communication of a lead's characteristics. Although the use of the implantable device has been described for use as a lead in, for example, a cardiac stimulation system, the implantable device could as well be applied to other types of body stimulating systems. Many other embodiments will be apparent to those of skill in the art upon reviewing the above description. The scope should, therefore, be determined with reference to the appended claims, along with the full scope of equivalents to which such claims are entitled.

I claim:

1. An implantable lead, comprising:
a body; and
indicia associated with the body, the indicia being adapted to indicate, external to a patient's body, operational characteristics of the lead.

2. The lead of claim 1, wherein the operational characteristics include at least one mechanical property of the lead.

3. The lead of claim 2, wherein the stiffness is one selected from a group of bending stiffness and torsional stiffness.

4. The lead of claim 2, wherein the mechanical property includes weight.

5. The lead of claim 1, wherein the operational characteristics include lubricity of the lead.

6. The lead of claim 1, wherein the operational characteristics include at least one electrical property of the lead.

7. The lead of claim 1, wherein the indicia is on an outer surface of the body.

8. The lead of claim 7, wherein the indicia include a plurality of alpha-numeric characters which represent a corresponding plurality of lead characteristics.

9. The lead of claim 1, wherein the indicia include a graphic representation of the characteristics of the lead.

10. The lead of claim 9, wherein the graphic representation includes a grid.

11. The lead of claim 10, wherein the grid is a three-dimensional grid having a plurality of locations each corresponding to a specific characteristic of the lead.

12. The lead of claim 9, wherein the graphic representation includes a bar graph.

13. The lead of claim 12, wherein the bar graph includes a bar representing a lead characteristic variable, and the bar has a height representing a value of the lead characteristic variable.

14. The lead of claim 1, wherein the body includes electrical conductor, an insulator surrounding the electrical conductor, and a coating on the insulator.

15. The lead of claim 14, wherein the indicia is beneath the coating.

16. The lead of claim 15, wherein the coating is transparent over the indicia.

17. The lead of claim 15, wherein the coating includes a therapeutic agent.

18. The lead of claim 1, wherein the indicia includes a first indicia denoting operational characteristics of a first portion of the body and a second indicia denoting operational characteristics of a second portion of the body.

19. The lead of claim 1, wherein the indicia indicates a statistical representation of operational characteristics of a first portion of the body and operational characteristics of a second portion of the body.

20. The lead of claim 1, wherein the indicia indicates an average of the operational characteristics of the body.

21. An apparatus, comprising:
a display;
indicia associated with the display, the indicia representing operational characteristics of an implantable lead.

22. The apparatus of claim 21, wherein the display includes an electronic device.

23. The apparatus of claim 22, wherein the electronic device includes a computer readable media storing the indicia and data related to the characteristics denoted by the indicia.

24. The apparatus of claim 22, wherein the electronic device includes a computer network.

25. The apparatus of claim 24, wherein the computer network includes a unit for entering a request for the indicia, and a server operably connected to the unit and adapted to provide the indicia to the unit.

26. The apparatus of claim 25, wherein the server is adapted to provide additional data associated with the indicia.

27. The apparatus of claim 21, wherein the indicia is adapted to indicate at least one of a mechanical characteristic and an electrical characteristic of an implantable lead.

28. The apparatus of claim 27, wherein the mechanical characteristic includes at least one of bending stiffness, torsional stiffness, and weight.

29. The apparatus of claim 21, wherein the indicia is on the display.

30. The apparatus of claim 21, wherein the display is adapted to be connected to an implantable lead.

31. The apparatus of claim 30, wherein the indicia include a graphical representation of lead operational characteristics.

32. The apparatus of claim 30, wherein the indicia include an alpha-numeric code representing lead operational characteristics.

33. The apparatus of claim 30, wherein the indicia are stored in a machine readable medium remotely from the lead.

34. A method for assigning indicia to an implantable lead, comprising:
providing characteristics of a lead type;
assigning indicia representing the characteristics of the lead type; and
associating the assigned indicia to leads of the lead type.

35. The method of claim 34, wherein associating includes imprinting the indicia on the lead.

36. The method of claim 34, wherein associating includes attaching the indicia to the lead.

37. The method of claim 34, wherein assigning includes assigning an alpha-numeric code for the indicia.

38. The method of claim 34, wherein associating includes storing the indicia in a computer readable media.

39. The method of claim 38, wherein associating includes providing access to the computer readable media.

40. The method of claim 39, wherein providing access includes providing access to the indicia through a computer network.

41. The method of claim 40, wherein associating includes storing additional data relating to the indicia and the lead type in the computer readable media.

42. The method of claim 41, wherein providing access includes requesting the user to select the indicia before providing access to the additional information.
